(19) United States
(12) Patent Application Publication
(10) Pub. No.: US 2025/0235454 A1
(43) Pub. Date: Jul. 24, 2025

(54) XOPHOS INHIBITORS FOR USE IN THE TREATMENT OF B-CELL LYMPHOMA

(71) Applicants: CENTRE NATIONAL DE LA RECHERCHE SCIENTIFIQUE, PARIS (FR); UNIVERSITE CLAUDE BERNARD LYON 1, Villeurbanne (FR); INSTITUT NATIONAL DE LA SANTE ET DE LA RECHERCHE MEDICALE, PARIS (FR); CENTRE LEON BERARD, LYON (FR); UNIVERSITE DE CAEN NORMANDIE, CAEN (FR)

(72) Inventors: Marc BILLAUD, Nice (FR); Martine CORDIER-BUSSAT, CHASSIEU (FR); Peggy SUZANNE, CAIRON (FR); Patrick DALLEMAGNE, SEULLINE (FR); Samir YAHIAOUI, BISCHHEIM (FR)

(73) Assignees: CENTRE NATIONAL DE LA RECHERCHE SCIENTIFIQUE, PARIS (FR); UNIVERSITE CLAUDE BERNARD LYON 1, Villeurbanne (FR); INSTITUT NATIONAL DE LA SANTE ET DE LA RECHERCHE MEDICALE, PARIS (FR); CENTRE LEON BERARD, LYON (FR); UNIVERSITE DE CAEN NORMANDIE, CAEN (FR)

(21) Appl. No.: 18/853,766

(22) PCT Filed: Apr. 7, 2023
(86) PCT No.: PCT/EP2023/059306

§ 371 Date: Oct. 3, 2024

(30) Foreign Application Priority Data
Apr. 8, 2022 (FR) FR2203261

(51) Int. Cl. A61K 31/506; A61K 31/496; A61P 35/00; C07D 213/74; C07D 239/42; C07D 403/12

(52) U.S. Cl. CPC A61K 31/506; A61K 31/496; A61P 35/00; C07D 213/74; C07D 239/42; C07D 403/12

(57) ABSTRACT

A compound derived from an aliphatic diamine including at least one pyridine or pyrimidine unit, for use as an anticancer agent, to a therapeutic composition including this compound, to a product including such a compound and another active agent, as well as to such a compound.

The present invention applies to the therapeutic field of cancer. In particular, the invention is directed to a compound derived from an aliphatic diamine comprising at least one pyridine or pyrimidine unit, for use as an anticancer agent, to a therapeutic composition comprising said compound, to a product comprising such a compound and another active agent, as well as to such a compound.

Cancer represents one of the leading causes of death worldwide. Treatments for cancer are varied and include surgery, radiotherapy, chemotherapy, hormone therapy, immunotherapy and targeted therapy. Fundamental research data shows that the plasticity of tumour cells allows them to develop resistance mechanisms in order to escape these treatments.

In this context, faced with the relatively low efficacy of the vast majority of traditional anti-cancer drugs in the treatment of cancers that have failed after first-line treatment or not, such as T-cell lymphomas, pancreatic adeno-carcinomas and some paediatric brain tumours, research is focusing on new therapeutic strategies. Indeed, successfully overcoming the problems of resistance encountered in this type of disease represents a real public health issue and a challenge for research.

Reconfiguration of energy metabolism is one of the key steps involved in tumour development, particularly in cases of resistance to treatment. Especially, tumour cells adapt to the conditions of their microenvironment and to the selective pressure exerted by chemotherapeutic treatments by adjusting their metabolism. The development of new molecules targeting cellular metabolism thus represents a major therapeutic challenge.

Mitochondria are organelles that play a key role in cellular metabolism by centralising ATP production from numerous substrates via oxidative phosphorylation (OXPHOS). Enzymatic reactions involved in this process regulate cell proliferation, differentiation, activation and self-renewal. Numerous recent studies have demonstrated a correlation between OXPHOS activity (i.e. activation of mitochondrial metabolism) and chemoresistance and/or tumour progression. In particular, mitochondria are cellular organelles capable of integrating and relaying multiple signals and contributing not only to the production of energy as ATP but also to the synthesis of macromolecules essential for tumour proliferation. Adaptation towards increased OXPHOS activity is a characteristic often acquired during tumour progression, especially during resistance to chemotherapy.

Molecules targeting OXPHOS metabolism by various processes have been developed and are being tested in various clinical trials; for example those blocking the mitochondrial ribosome and indirectly the synthesis of respiratory chain complexes (e.g. the antibiotic tigecycline), or those directly inhibiting complex I (e.g. metformin) or complex III (e.g. antimycin A) of the respiratory chain. They have a synergistic cytotoxic effect with reference treatments. Other pharmacological approaches aimed at blocking β-oxidation of fatty acids in mitochondria or increasing oxidative stress in OXPHOS malignant cells have also been provided.

More recently, other OXPHOS inhibitors have been described. In particular, a complex I inhibitor known as “IACS-010759” is currently undergoing clinical trials, especially in haematological cancers.

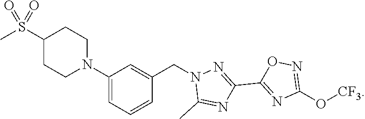 [SMILES: Cc1nc(-c2nc(OC(F)(F)F)no2)nn1Cc1cccc(N2CCC(S(C)(=O)=O)CC2)c1]

In addition, international application WO2020/109506 describes compounds having the following formula:

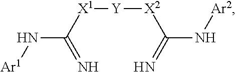

wherein X¹ and X², which are identical or different, are NR⁵ or a sulphur atom, Y is a C₁-C₁₀ alkanediyl group, Ar¹ and Ar², which are identical or different, are an optionally substituted aryl group, and R⁵ is a hydrogen atom or a C₁-C₆ alkyl group, or a pharmaceutically acceptable salt and/or solvate thereof, for use in cancer treatment. These compounds are described as inhibitors of oxygen consumption rate by mitochondria, and would especially enable treatment of some cancers having an “OXPHOS” mechanism.

However, in order to meet the growing need for personalised medicine, based on the individual properties of each tumour, there is a persistent need to develop other anti-cancer agents that are effective on tumour cells, in particular those targeting the mitochondrial respiratory chain, and therefore having an inhibitory effect on the cell energy charge. In particular, there is a need to develop molecules with other physicochemical characteristics, acting on other intracellular targets compared with the limited number of OXPHOS inhibitors already developed. These new molecules should be easy to prepare, and have improved cytotoxicity properties while guaranteeing good pharmacokinetic properties such as good ADMET properties (Absorption of the molecule, Distribution in the body, Elimination including biotransformation or Metabolism, and Excretion, and Toxicity), especially in silico.

Thus, the purpose of the present invention is to overcome drawbacks of the aforementioned prior art and to provide an anti-cancer agent with good performance in terms of anti-cancer activity, which is easy to prepare and has low toxicity on non-tumour cells while guaranteeing good ADMET properties.

The purpose of the invention is achieved by the compounds that will be described below.

The first object of the present invention is a compound selected from compounds having the formula (I), pharmaceutically acceptable salts thereof, and pharmaceutically acceptable solvates thereof,
said formula (I) having the following structure:

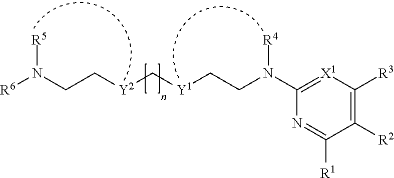

wherein:
   R¹, R², and R³ represent, independently of each other, a hydrogen atom, a halogen atom, an alkyl radical, a cycloalkyl radical, an aryl radical, or a group selected from —OH, —NH₂, —SH, —CN (cyano or carbonitrile), CF₃, —CHO (aldehyde), —NH—NH₂ (hydrazine), —CO₂H, —CH₂OH, CH₂NH₂, an alkoxy group —OR⁷, a —NR⁸R⁹ group, a —SR¹⁰ group, a —C(O)R¹¹ group, a —CH₂OR¹² group and a —CH₂NR¹³R¹⁴ group, with R⁷, R⁸, R¹⁰, R¹¹, R¹², and R¹³ representing, independently of each other, an alkyl or cycloalkyl radical, and R⁹ and R¹⁴ representing, independently of each other, a hydrogen atom or an alkyl or cycloalkyl radical,
X¹ represents a nitrogen atom or a CR¹⁵ group, with R¹⁵ being a hydrogen atom, a halogen atom, an alkyl radical, a cycloalkyl radical or an aryl radical, or a group selected from —OH, —NH₂, —SH, —CN, —CF₃, —CHO, —NH—NH₂, —CO₂H, —CH₂OH, —CH₂NH₂, an alkoxy group —OR⁷, a —NR⁸R⁹ group, a —SR¹⁰ group, a —C(O)R¹¹ group, a —CH₂OR¹² group, and a —CH₂OR¹² group, and a —CH₂NR¹³R¹⁴ group,
n is an integer ranging from 1 to 20,
R⁴ represents a hydrogen atom, an alkyl radical, or a divalent alkylene group forming a ring with Y¹ and the nitrogen atom to which R⁴ is attached,
R⁵ represents a hydrogen atom, an alkyl radical, or a divalent alkylene group forming a ring with Y² and the nitrogen atom to which R⁵ is attached,
Y¹ represents —CH₂—, —NH—, or —O— when the R⁴ group represents a hydrogen atom or an alkyl radical; and represents —CH— or —N— when the R⁴ group represents a divalent alkylene group forming a ring with Y¹ and the nitrogen atom to which R⁴ is attached,
Y² represents —CH₂—, —NH—, or —O— when the R⁵ group represents a hydrogen atom or an alkyl radical; and represents —CH— or —N— when the R⁵ group represents a divalent alkylene group forming a ring with Y² and the nitrogen atom I to which R⁴ is attached, and
R⁶ represents one of the following two groups (IIa) and (IIb):

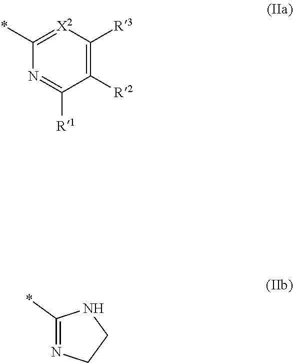

wherein R′¹, R′², and R′³ represent, independently of each other, a hydrogen atom, a halogen atom, an alkyl radical, a cycloalkyl radical, an aryl radical, or a group selected from —OH, —NH₂, —SH, CN, CF₃, —CHO, —NH—NH₂, —CO₂H, —CH₂OH, —CH₂NH₂, an alkoxy group —OR′⁷, a —NR′⁸R′⁹ group, a —SR′¹⁰ group, a —C(O)R′¹¹ group, a —CH₂OR′¹² group, and a —CH₂NR′¹³R′¹⁴ group, with R′⁷, R′⁸, R′¹⁰, R′¹¹, R′¹², and R′¹³ representing, independently of each other, an alkyl or cycloalkyl radical, and R′⁹ and R′¹⁴ representing, independently of each other, a hydrogen atom or an alkyl or cycloalkyl radical, and
X² represents a nitrogen atom or a CR′¹⁵ group, with R′¹⁵ being a hydrogen atom, a halogen atom, an alkyl radical, a cycloalkyl radical, an aryl radical, or a group selected from —OH, —NH₂, —SH, —CN, —CF₃, —CHO, —NH—NH₂, —CO₂H, —CH₂OH, —CH₂NH₂, an alkoxy group —OR′⁷, a —NR′⁸R′⁹ group, a —SR′¹¹ group, a —C(O)R′¹¹ group, a —CH₂OR′¹² group, and a —CH₂NR′¹³R′¹⁴ group,
for use in cancer treatment.

Compounds (I) of the invention are derivatives of an aliphatic diamine comprising at least one pyridine or pyrimidine unit. The inventors have discovered that such compounds exhibit significant anti-cancer activity. In addition, these compounds are easy to prepare, have low toxicity for healthy cells, and good ADMET properties, especially in silico.

By “cancer”, it is meant all malignant neoplastic formations, whatever their histological nature (adult and paediatric). There are two main categories of solid tumours: carcinomas, of epithelial origin, and sarcomas, of connective origin. Solid tumours are formed by atypical cells that are invasive or likely to disseminate, generally characterised by an autonomous capacity for growth, an imprecise delineation, a capacity to invade neighbouring tissues and vessels and a tendency to disseminate through the production of metastases. Examples include breast, prostate, lung, oesophagus, skin, bladder, stomach, liver, uterus, colon and rectum cancers. There are also pancreatic endocrine and exocrine cancers, and paediatric tumours such as rhabdomyosarcomas and Diffuse Intrinsic Pontine Gliomas (DIPG). The other category of tumours includes the various types of haematological malignancies.

The second object of the invention is a compound as defined in the first object of the invention, for targeted use in the treatment of cancers with altered metabolism, in particular in the treatment of cancers with OXPHOS metabolism.

A cancer with an OXPHOS metabolism corresponds to a cancer which includes or is constituted by cancer cells mainly relying on oxidative phosphorylation (OXPHOS) for biosynthetic and/or bioenergetic processes.

Such cancers with OXPHOS metabolism include haematological cancers, lung cancers, cervical cancers, prostate cancers, neuroendocrine tumours, glial tumours and skin and eye cancers.

According to a preferred embodiment of the invention, the compound as defined in the first object of the invention is used in the treatment of lymphomas, especially adult or paediatric B and T lymphomas; solid tumours such as sarcomas, especially paediatric sarcomas (e.g. of the rhabdomyosarcoma type); and some recurrent tumours following chemotherapy treatments.

The compound as defined in the first object of the invention has antitumour activity in one or several preclinical models.

By “lymphoma”, it is meant any tumour, usually malignant, caused by a proliferation of lymphoid tissue cells, developing in the spleen or lymph nodes, but also in many other organs or tissues.

The compound as defined in the first object of the invention thus has the potential properties of a new anti-cancer drug targeting cellular metabolism.

It acts especially as an inhibitor of the mitochondrial respiratory chain and inhibits oxygen consumption rate. It can thus be considered an OXPHOS inhibitor. It also has:
in vitro cytotoxic activity competitive with OXPHOS inhibitors already in clinical trials, in particular IACS-010759, and
specificity of action related to the cellular phenotype.

In the compound as defined in the first object of the invention, the alkyl radical may be linear or branched, and is preferably linear.

In the compound as defined in the first object of the invention, the cycloalkyl radical may be linear or branched, and is preferably linear.

For the purposes of the present invention, a halogen is selected from F, Cl, Br and I, and particularly preferred from F and Cl.

### Definition of R¹, R² R³

R¹, R², and R³ represent, independently of each other, a hydrogen atom, a halogen atom, an alkyl radical, a cycloalkyl radical, an aryl radical, or a group selected from —OH, —NH₂, —SH, —CN (carbonitrile or cyano), —CF₃, —CO₂H, —CH₂OH, —CH₂NH₂, —CHO (aldehyde), —NH—NH₂ (hydrazine), an alkoxy group —OR⁷, a —NR⁸R⁹ group, a —SR′¹⁰ group, a —C(O)R¹¹ group, a —CH₂OR¹² group, and a —CH₂NR¹³R¹⁴ group, with R⁷, R⁸, R¹⁰, R¹¹, R¹², and R¹³ representing, independently of each other, an alkyl or cycloalkyl radical, and R⁹ and R¹⁴ represent, independently of each other, a hydrogen atom or an alkyl or cycloalkyl radical.

The alkyl radical as the R¹, R² or R³ group is preferably a C₁-C₅ alkyl radical, and particularly preferably a methyl, ethyl, propyl, isopropyl, butyl or tert-butyl radical.

The cycloalkyl radical as the R¹, R² or R³ group is preferably a C₃-C₆ cycloalkyl radical, particularly preferably a cyclopropyl, cyclobutyl, cyclopentyl or cyclohexyl radical, and more particularly preferably a cyclopropyl radical.

The aryl radical as the R¹, R² or R³ group is preferably a C₅-C₁₅ aryl radical, particularly preferably a phenyl, 2- or 3-thienyl, 2- or 3-furyl radical, and more particularly preferably a phenyl radical.

### Definition of R⁷, R⁸, R⁹, R¹⁰, R¹¹, R¹², R¹³ and R¹⁴

The alkyl radical as the R⁷, R⁸, R⁹, R¹⁰, R¹¹, R¹², R¹³ or R¹⁴ group is preferably a C₁-C₅ alkyl radical, and particularly preferably a methyl, ethyl, propyl, isopropyl, butyl or tert-butyl radical.

The cycloalkyl radical as the R⁷, R⁸, R⁹, R¹⁰, R¹¹, R¹², R¹³ or R¹⁴ group is preferably a C₃-C₆ cycloalkyl radical, particularly preferably a cyclopropyl, cyclobutyl, cyclopentyl or cyclohexyl radical, and more particularly preferably a cyclopropyl radical.

The —SR¹⁰ group is preferably a thiomethyl group.

The —NR⁸R⁹ group is preferably a methylamine or dimethylamine group.

The alkoxy group —OR⁷ is preferably a methoxy or ethoxy group.

According to a particularly preferred embodiment of the invention, R¹ is a hydrogen atom, a halogen atom, an alkyl radical, a cycloalkyl radical, an alkoxy group —OR⁷, a —SR¹⁰ group, a —CN group, a CF₃ group, a —NR⁸R⁹ group, a —NH—NH₂ group, a —CO₂H group or a —CHO group.

According to a particularly preferred embodiment of the invention, R² is a hydrogen atom, a halogen atom, an alkyl radical, a cycloalkyl radical, an alkoxy group —OR⁷, a —SR¹⁰ group, a —CN group, a CF₃ group, a —NR⁸R⁹ group, a —NH—NH₂ group, a —CO₂H group or a —CHO group.

According to a particularly preferred embodiment of the invention, R³ is a hydrogen atom, a halogen atom, an alkyl radical, a cycloalkyl radical, an alkoxy group —OR⁷, a —SR¹⁰ group, a —CN group, a CF₃ group, a —NR⁸R⁹ group, a —NH—NH₂ group, a —CO₂H group or a —CHO group.

Advantageously, R¹, R² and R³ are hydrogen atoms.

### Definition of X¹

X¹ represents a nitrogen atom or a CR¹⁵ group, with R¹⁵ being a hydrogen atom, a halogen atom, an alkyl radical, a cycloalkyl radical or an aryl radical, or a group selected from —OH, —NH₂, —SH, —CN, —CF₃, —CO₂H, —CH₂OH, —CH₂NH₂, an alkoxy group —OR⁷, a —NR⁸R⁹ group, a —SR¹⁰ group, a —C(O)R¹¹ group, a —CH₂OR¹² group, and a —CH₂NR¹³R¹⁴ group.

The R⁷, R⁸, R⁹, R¹⁰, R¹¹, R¹², R¹³ and R¹⁴ groups are as defined in the invention.

According to a particularly preferred embodiment of the invention, R¹⁵ is a hydrogen atom, a halogen atom, an alkyl radical, a cycloalkyl radical, an alkoxy group —OR⁷, a —SR¹⁰ group, a —CN group, a —CF₃ group, a —NR⁸R⁹ group, a —NH—NH₂ group, a —CO₂H group, or a —CHO group.

Advantageously, X¹ represents a nitrogen atom or a CH group.

### Definition of R⁴ and R⁵

R⁴ represents a hydrogen atom, an alkyl radical, or a divalent alkylene group forming a ring with Y¹ and the nitrogen atom to which R⁴ is attached.

The alkyl radical as the R⁴ group is preferably a C₁-C₅ alkyl radical, and particularly preferably a methyl or ethyl radical.

The divalent alkylene radical forming a ring with Y¹ and the nitrogen atom to which R⁴ is attached as the group R⁴ is preferably an alkylene radical —(CH₂)₂— or —(CH₂)₃—, and particularly preferably an alkylene radical —(CH₂)₂—.

Preferably, R⁴ represents a hydrogen atom or a divalent alkylene group forming a ring with Y¹ and the nitrogen atom to which R⁴ is attached.

R⁵ represents a hydrogen atom, an alkyl radical, or a divalent alkylene group forming a ring with Y² and the nitrogen atom to which R⁵ is attached.

The alkyl radical as the R⁵ group is preferably a C₁-C₅ alkyl radical, and particularly preferably a methyl, ethyl, propyl or butyl radical.

The divalent alkylene radical forming a ring with Y² and the nitrogen atom to which R² is attached as the R² group is preferably an alkylene radical —(CH₂)₂— or —(CH₂)₃—, and particularly preferably an alkylene radical —(CH₂)₂—.

Preferably, R⁵ represents a hydrogen atom or a divalent alkylene group forming a ring with Y² and the nitrogen atom to which R⁵ is attached.

According to a particularly preferred embodiment of the invention, R⁴ represents a hydrogen atom or a divalent alkylene group forming a ring with Y¹ and the nitrogen atom to which R⁴ is attached; and R⁵ represents a hydrogen atom or a divalent alkylene group forming a ring with Y² and the nitrogen atom to which R⁵ is attached.

According to a particularly preferred embodiment of the invention, R⁴ and R⁵ are identical.

### Definition of Y¹ and Y²

Y¹ represents —CH₂—, —NH— or —O— when the R⁴ group represents a hydrogen atom or an alkyl radical; and represents —CH— or —N— when the R⁴ group represents a divalent alkylene group forming a ring with Y¹ and the nitrogen atom to which R⁴ is attached.

Preferably, Y¹ represents —CH₂— when the R⁴ group represents a hydrogen atom or an alkyl radical; and represents —N— when the R⁴ group represents a divalent alkylene group forming a ring with Y¹ and the nitrogen atom to which R⁴ is attached.

Y² represents —CH₂—, —NH— or —O— when the R⁵ group represents a hydrogen atom or an alkyl radical; and represents —CH— or —N— when the R⁵ group represents a divalent alkylene group forming a ring with Y² and the nitrogen atom to which R⁵ is attached.

Preferably, Y² represents —CH₂— when the R⁵ group represents a hydrogen atom or an alkyl radical; and represents —N— when the R⁵ group represents a divalent alkylene group forming a ring with Y² and the nitrogen atom to which R⁵ is attached.

According to a particularly preferred embodiment of the invention, Y¹ represents —CH₂— when the R⁴ group represents a hydrogen atom or an alkyl radical; and represents —N— when the R⁴ group represents a divalent alkylene group forming a ring with Y¹ and the nitrogen atom to which R⁴ is attached; and Y² represents —CH₂— when the R⁵ group represents a hydrogen atom or an alkyl radical; and represents —N— when the R⁵ group represents a divalent alkylene group forming a ring with Y² and the nitrogen atom to which R⁵ is attached.

According to a particularly preferred embodiment of the invention, Y¹ and Y² are identical.

### Definition of n

n is an integer ranging from 1 to 20, and preferably from 1 to 14.

### Definition of Y¹, Y² in Relation to n

According to a preferred embodiment of the invention:
when Y¹ (respectively Y²) represents —CH₂—, n preferably ranges from 2 to 10, and particularly preferably from 3 to 9,
when Y¹ (respectively Y²) represents —N—, —NH—, —CH— or —O—, n preferably ranges from 6 to 18, and particularly preferably from 7 to 14.

This embodiment is particularly suitable when R⁶ is a group of formula (IIa).

When Y¹ and Y² represent —CH—, and R⁶ is a group of the formula (IIa), then n is preferably such that n≥5 and/or R¹, R′¹, R², R′², R³, and R′³ are preferably different from —NH₂.

When Y¹ and Y² represent —N—, and R⁶ is a group of formula (IIa), then n is preferably such that n≥7.

According to a preferred embodiment of the invention, when Y¹ and Y² represent —CH—, and R⁶ is a group of formula (IIa), then n is such that n≥5 and/or R¹, R′¹, R², R′², R³, and R′³ are different from —NH₂; and when Y¹ and Y² represent —N—, and R⁶ is a group of formula (IIa), then n is such that n≥7.

### Definition of R⁶

R⁶ represents one of the following two groups (IIa) and (IIb):

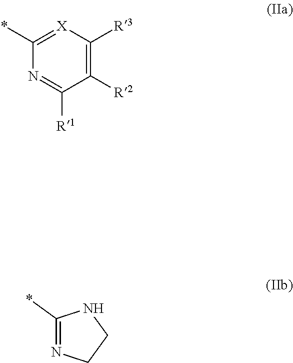

wherein R′¹, R′², and R′³ represent, independently of each other, a hydrogen atom, a halogen atom, an alkyl radical, a cycloalkyl radical, an aryl radical, or a group selected from —OH, —NH₂, —SH, —CN, CF₃, —CHO, —NH—NH₂, —CO₂H, —CH₂OH, —CH₂NH₂, an alkoxy group —OR′⁷, a —NR′⁸R′⁹ group, a —SR′¹⁰ group, a —C(O)R′¹¹ group, a —CH₂OR¹² group, and a —CH₂NR′¹³R′¹⁴ group, with R′⁷, R′⁸, R′¹⁰, R′¹¹, R′¹², and R′¹³ representing, independently of each other, an alkyl or cycloalkyl radical, and R′⁹ and R′¹⁴ representing, independently of each other, a hydrogen atom, or an alkyl or cycloalkyl radical, and
X² represents a nitrogen atom or a CR′¹⁵ group, with R′¹⁵ being a hydrogen atom, a halogen atom, an alkyl radical, a cycloalkyl radical, an aryl radical, or a group selected from —OH, NH₂, —SH, —CN, —CF₃, —CO₂H, —CH₂OH, —CH₂NH₂, an alkoxy group —OR′⁷, a —NR′⁸ R′⁹ group, a —SR′¹¹ group, a —C(O)R′¹¹ group, a —CH₂OR′¹² group, and a —CH₂NR′¹³R′¹⁴ group.
R⁶ is preferably a group of formula (IIb).

### Definition of R′¹, R′², R′³

The alkyl radical as the R′¹, R′² or R′³ group is preferably a C₁-C₅ alkyl radical, and particularly preferably a methyl, ethyl, propyl, isopropyl, butyl or tert-butyl radical.

The cycloalkyl radical as the R′¹, R′² or R′³ group is preferably a C₃-C₆ cycloalkyl radical, and particularly preferably a cyclopropyl, cyclobutyl, cyclopentyl or cyclohexyl radical, and more particularly preferably a cyclopropyl radical.

The aryl radical as the R′¹, R′² or R′³ group is preferably a C₅-C₁₅ aryl radical, and particularly preferably a phenyl, 2- or 3-thienyl, 2- or 3-furyl radical, and more particularly preferably a phenyl radical.

### Definition of R′⁷, R′⁸, R′⁹, R′¹⁰, R′¹¹, R′¹², R′¹³ and R′¹⁴

The alkyl radical as the group R′⁷, R′⁸, R′⁹, R′¹⁰, R′¹¹, R′¹², R′¹³ or R′¹⁴ is preferably a C₁-C₅ alkyl radical, and particularly preferably a methyl, ethyl, propyl, isopropyl, butyl or tert-butyl radical.

The cycloalkyl radical as the group R′⁷, R′⁸, R′⁹, R′¹⁰, R′¹¹, R′¹², R′¹³ or R′¹⁴ is preferably a C₃-C₆ cycloalkyl radical, and particularly preferably a cyclopropyl, cyclobutyl, cyclopentyl or cyclohexyl radical, and more particularly preferably a cyclopropyl radical.

The —SR′¹⁰ group is preferably a thiomethyl group.

The —NR′⁸R′⁹ group is preferably a methylamine or dimethylamine group.

The alkoxy group —OR′⁷ is preferably a methoxy or ethoxy group.

According to a particularly preferred embodiment of the invention, R′¹ is a hydrogen atom, a halogen atom, an alkyl radical, a cycloalkyl radical, an alkoxy group —OR⁷, a —SR¹⁰ group, a —CN group, a CF₃ group, a —NR⁸R⁹ group, a —NH—NH₂ group, a —CO₂H group or a —CHO group.

According to a particularly preferred embodiment of the invention, R′² is a hydrogen atom, a halogen atom, an alkyl radical, a cycloalkyl radical, an alkoxy group —OR⁷, a —SR¹⁰ group, a —CN group, a CF₃ group, a —NR⁸R⁹ group, a —NH—NH₂ group, a —CO₂H group or a —CHO group.

According to a particularly preferred embodiment of the invention, R′³ is a hydrogen atom, a halogen atom, an alkyl radical, a cycloalkyl radical, an alkoxy group —OR⁷, a-SR¹⁰ group, a —CN group, a CF₃ group, a —NR⁸R⁹ group, a —NH—NH₂ group, a —CO₂H group or a —CHO group.

Advantageously, R′¹, R′² and R′³ are hydrogen atoms.

### Definition of X²

X² represents a nitrogen atom or a CR′¹⁵ group, with R′¹⁵ being a hydrogen atom, a halogen atom, an alkyl radical, a cycloalkyl radical, an aryl radical, or a group selected from —OH, NH₂, —SH, —CN, —CF₃, —CO₂H, —CH₂OH, —CH₂NH₂, an alkoxy group —OR′⁷, a —NR′⁸R′⁹ group, a —SR′¹⁰ group, a —C(O)R′¹¹ group, a —CH₂OR′¹² group, and a —CH₂NR′¹³R′¹⁴ group.

The R′⁷, R′⁸, R′⁹, R′¹⁰, R′¹¹, R′¹², R′¹³ and R′¹⁴ groups are as defined in the invention.

According to a particularly preferred embodiment of the invention, R′¹⁵ is a hydrogen atom, a halogen atom, an alkyl radical, a cycloalkyl radical, an alkoxy group —OR⁷, a —SR′¹⁰ group, a —CN group, a —CF₃ group, a —NR⁸R⁹ group, a —NH—NH₂ group, a —CO₂H group or a —CHO group.

Advantageously, X² represents a nitrogen atom or a CH group.

According to a preferred embodiment of the invention, the compound of formula (I) is selected from the following compounds:

| Name | Structural formula |
|---|---|
| Compound Ia | 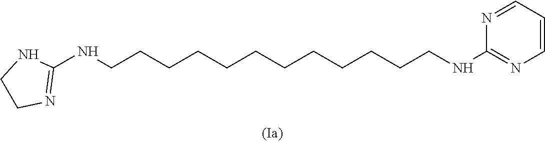 [SMILES: c1cnc(NCCCCCCCCCCCCNC2=NCCN2)nc1] |
| Compound Ib | 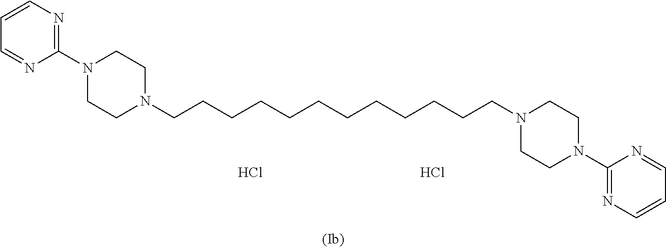 [SMILES: c1cnc(N2CCN(CCCCCCCCCCCCN3CCN(c4ncccn4)CC3)CC2)nc1 ; Cl ; Cl] |
| Compound Ic | 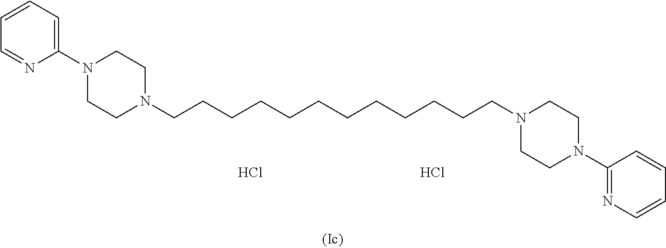 [SMILES: c1ccc(N2CCN(CCCCCCCCCCCCN3CCN(c4ccccn4)CC3)CC2)nc1 ; Cl ; Cl] |
| Compound Id | 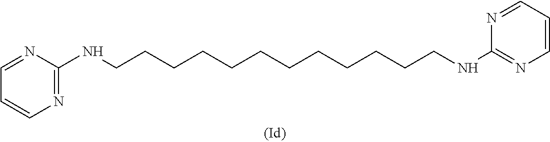 [SMILES: c1cnc(NCCCCCCCCCCCCNc2ncccn2)nc1] |
| Compound Ie | 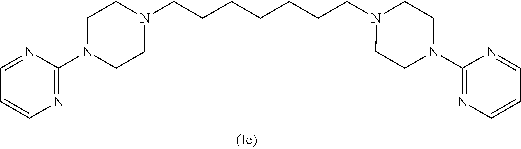 [SMILES: c1cnc(N2CCN(CCCCCCCN3CCN(c4ncccn4)CC3)CC2)nc1] |
| Compound If | 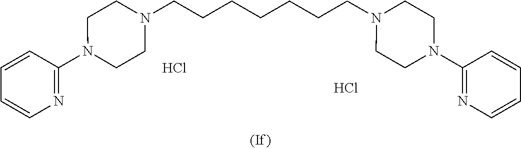 [SMILES: c1ccc(N2CCN(CCCCCCCN3CCN(c4ccccn4)CC3)CC2)nc1 ; Cl ; Cl] |

The term “pharmaceutically acceptable” means useful for the preparation of a pharmaceutical composition and generally safe and non-toxic for pharmaceutical use.

By pharmaceutically acceptable salt or solvate of a compound, it is meant a salt or solvate which is pharmaceutically acceptable as defined above, and which possesses the pharmacological activity of said compound.

Pharmaceutically acceptable salts include:
Acid addition salts formed with inorganic acids such as hydrobromic acid, hydrochloric acid, sulphuric acid, nitric acid or phosphoric acid; or formed with organic acids such as formic acid, acetic acid, benzenesulphonic acid, fumaric acid, glucoheptonic acid, gluconic acid, glutamic acid, glycolic acid, hydroxynaphthoic acid, 2-hydroxyethane sulphonic acid, maleic acid, malic acid, mandelic acid, methanesulphonic acid, propionic acid, succinic acid, muconic acid, 2-naphthalenesulphonic acid, tartaric acid, dibenzoyl-L-tartaric acid, paratoluenesulphonic acid, trimethylacetic acid, trifluoroacetic acid, benzoic acid, citric acid, ethanesulphonic acid, lactic acid, mucic acid, pamoic acid or pantothenic acid,
salts formed when an acidic proton present in the compound is either replaced with a metal ion, such as an alkali metal, alkaline earth metal, or aluminium ion; or coordinated with an inorganic or organic base. Acceptable organic bases include diethanolamine, ethanolamine, N-methylglucamine, triethanolamine, and tromethamine. Acceptable inorganic bases include aluminium hydroxide, calcium hydroxide, potassium hydroxide, sodium carbonate and sodium hydroxide.

Solvates acceptable for therapeutic use of the compounds of the invention include conventional solvates such as those formed during the last step of preparing these compounds due to the presence of solvents. Solvates related to the presence of water (these solvates are also called hydrates) or ethanol may be mentioned.

Because of their anti-cancer activity, compounds as defined in the first object of the invention are useful in therapy.

The third object of the invention is a pharmaceutical composition comprising a compound as defined in the first object of the invention and at least one suitable pharmaceutical carrier.

The suitable pharmaceutical carrier may be a pharmaceutically acceptable excipient for use in the treatment of cancer, and in particular in the treatment of cancers with altered metabolism, and preferably cancers with OXPHOS metabolism.

The pharmaceutical composition may be a solid composition or a liquid composition.

The solid composition may be in the form of tablets, capsules, powders or granules.

The tablets may comprise the compound as defined in the first object of the invention in admixture with a pharmaceutical carrier such as gelatin, starch, lactose, magnesium stearate, talc, gum arabic or the like. The resulting mixture can in particular be compressed.

The tablets may be coated with saccharose, sucrose or other suitable materials, or may be treated so that they have prolonged or delayed activity and continuously release a predetermined amount of compound.

The powders or granules may be water dispersible. They may contain the compound as defined in the first object of the invention in admixture with dispersing agents, wetting agents or suspending agents, especially with taste adjusters or sweeteners.

The capsules may comprise the compound as defined in the first object of the invention in admixture with a diluent. The capsules may be soft or hard capsules.

The liquid composition may be in the form of an aqueous suspension or solution, a syrup or an elixir.

It may especially comprise the compound as defined in the first object of the invention in a solvent such as water, optionally as well as an appropriate sweetener, taste agent and/or colourant.

The liquid composition may especially be obtained by dissolving or suspending a powder or granules as aforesaid in a liquid such as water, fruit juice, milk, etc.

The pharmaceutical composition is preferably sterile. It may be in the form of an isotonic solution (particularly in comparison with blood).

The compound as defined in the first object of the invention or the pharmaceutical composition according to the third object of the invention may thus be used in a method for the therapeutic treatment of cancer, said method comprising administering to an individual an effective amount of said compound as defined in the first object of the invention (or of a pharmaceutically acceptable salt or solvate of said compound) or administering an effective amount of said pharmaceutical composition according to the third object of the invention.

The individual is the patient requiring treatment, such as a mammal, especially humans.

The compound or pharmaceutical composition can be administered to mammals, including humans, by the nasal, enteral (e.g. oral) or parenteral (e.g. intravenous) route.

Dosage varies according to the treatment and the condition in question. Suitable unit administration forms include oral forms such as tablets, capsules, powders, granules and oral solutions or suspensions, sublingual and buccal administration forms, subcutaneous, intramuscular, intravenous, intranasal or intraocular forms and rectal administration forms.

The compound as defined in the first object of the invention can be used alone in therapy, or in combination with at least one other active agent.

The present invention thus relates to a method for treating cancer in a patient in need thereof, comprising administering a compound as defined in the first object of the invention (or a pharmaceutically acceptable salt or solvate of said compound) or a pharmaceutical composition in accordance with the third object of the invention to the patient.

Cancer is as defined in the first object of the invention.

The present invention also relates to a method of treating a cancer with altered metabolism, in particular a cancer with OXPHOS metabolism, in a patient in need thereof, comprising administering a compound as defined in the first object of the invention (or a pharmaceutically acceptable salt or solvate of said compound) or a pharmaceutical composition in accordance with the third object of the invention to the patient.

Cancer with altered metabolism, in particular cancer with OXPHOS metabolism, are as defined in the first object of the invention.

The present invention also relates to the use of a compound as defined in the first object of the invention (or of a pharmaceutically acceptable salt or solvate of said compound) or of a pharmaceutical composition in accordance with the third object of the invention, for the manufacture of a drug intended for the treatment of a cancer or of a cancer with modified metabolism, in particular of a cancer with OXPHOS metabolism, in a subject in need thereof.

The present invention also relates to the use of a compound as defined in the first object of the invention (or of a pharmaceutically acceptable salt or solvate of said compound) or of a pharmaceutical composition in accordance with the third object of the invention, for treating a cancer or a cancer with altered metabolism, in particular a cancer with OXPHOS metabolism, in a subject in need thereof.

The term “patient” refers to an animal, generally a warm-blooded animal, preferably a mammal. The term “mammal” here refers to any mammal, including humans, domestic and farm animals, as well as zoo, sports or companion animals, such as dogs, cats, cattle, horses, sheep, pigs, goats, rabbits, etc. Preferably, the mammal is a primate, and more particularly a human being.

The term “human” refers to a male or female human subject at any stage of development, including the newborn, infant, juvenile, adolescent and adult.

The fourth object of the invention is a product comprising a compound as defined in the first object of the invention and another active agent.

The compound as defined in the first object of the invention and the other active agent are then used in combination, in particular for simultaneous, separate or staggered use in therapy.

These other active agents are in particular selected from those appropriate for the treatment of cancers. They may be adjuvants for improving activity of the compounds in accordance with the invention, or other active agents known for their use in the treatment of said conditions. Such active agents are well known to the person skilled in the art, are commercially available or are described in reference works such as Le Dictionnaire Vidal, which is published and updated every year, in particular the active agents grouped under the pharmacotherapeutic families “Cancerology Haematology”.

Certain compounds as defined in the first object of the invention are novel per se and represent the fifth object of the invention: these compounds are selected from compounds having the formula (I′), pharmaceutically acceptable salts thereof and pharmaceutically acceptable solvates thereof,
said formula (I′) having the following structure:

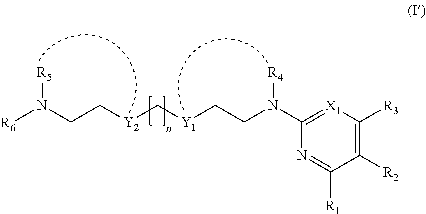

wherein:
   R¹, R², R³, R⁴, R⁵, R⁶, X¹, Y¹, Y², and n are as defined in the first object of the invention for formula (I), it being understood that:
   when Y¹ and Y² represent —CH—, and R⁶ is a radical of formula (IIa), then
   n≥5 and/or R′, R′¹, R², R′², R³, and R′³ are different from —NH₂, and
   when Y¹ and Y² represent —N—, and R⁶ is a radical of formula (IIa), then n≥7.

Further characteristics, alternatives and advantages of the compound, of its use, or of the pharmaceutical composition according to the invention will become clearer upon reading the following examples of embodiments, given by way of illustrating and not limiting purposes of the invention.

### BRIEF DESCRIPTION OF THE DRAWINGS

The attached drawings illustrate the invention.

FIG. 1 shows the oxygen consumption rate after treatment of cells from a human B cell lymphoma line with compounds in accordance with the invention and compounds from prior art.

FIG. 2 shows the effect on cell growth of treating human tumour cells from a B cell lymphoma line with compounds in accordance with the invention and compounds from prior art.

FIG. 3 shows the effect on cell growth of treating human tumour cells from a wide series of B and T cell lymphoma lines with a compound according to the invention and a compound from prior art.

FIG. 4 shows the anti-tumour efficacy of a compound of the invention in an in ovo model.

FIG. 5 shows the effect on cell growth of treating human cells from two paediatric sarcoma lines with compounds of the invention.

FIG. 6 shows the effect on cell growth of treating human tumour cells from a B cell lymphoma line with compounds in accordance with the invention.

FIG. 7 shows the effect on cell growth of treating human tumour cells from a paediatric sarcoma line with compounds of the invention.

### EXAMPLES

### Example 1: Synthesis of Compound Ia

Flash column chromatography has been performed on silica gel 60 (0.063200 mm).

Nuclear magnetic resonance spectra (¹H NMR and ¹³C NMR) have been recorded at 25° C. with a spectrometer (Bruker Avance III) (¹H NMR at 400 MHz, ¹³C NMR at 100 MHz) using CD₃OD as the reference solvent relative to residual CH₃OH (δH=3.31 ppm, δC=49.00±0.01 ppm) and DMSO-d₆ as the reference solvent relative to residual DMSO (δH=2.50 ppm, δC=39.52±0.06 ppm). Chemical shifts are given in ppm and coupling constants (J) in Hertz. Data for ¹H NMR spectra are reported as follows: ppm chemical shift (s=singlet, d=doublet, t=triplet, q=quadruplet, dd=doublet of doublets, td=triplet of doublets, ddd=doublet of doublets of doublets, m=multiplet, coupling constants, integration).

### Example 1: Synthesis of Compound Ia

Compound Ia has been prepared according to the steps illustrated in the following scheme:

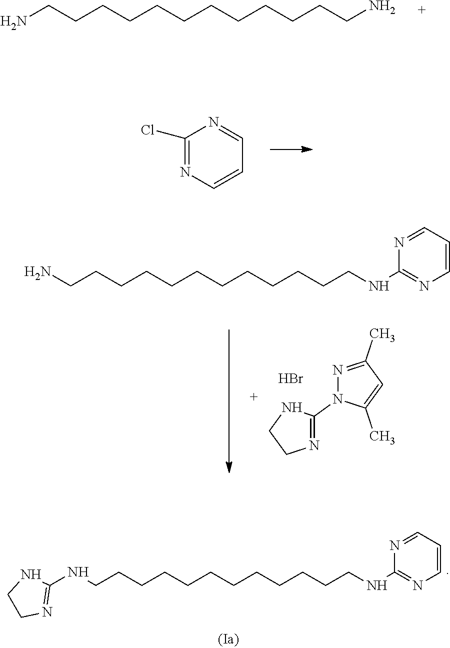 [SMILES: NCCCCCCCCCCCCN ; Clc1ncccn1 ; NCCCCCCCCCCCCNc1ncccn1 ; Cc1cc(C)n(C2=NCCN2)n1 ; Br ; c1cnc(NCCCCCCCCCCCCNC2=NCCN2)nc1]

First step: synthesis of the N′-pyrimidin-2-yldodecane-1,12-diamine derivative.

To a solution of 1 g (4.99 mmole) of 1,12-dodecanediamine in 30 mL of dioxane 0.8 equivalent (3.99 mmole; 457.2 mg) of 2-chloropyrimidine and 5 equivalents (24.95 mmole; 3.45 g) of potassium carbonate have been added. The reaction mixture has been heated under reflux of dioxane for 16 h. After cooling, the dioxane has been concentrated under reduced pressure. The residue obtained has been taken up in 50 mL of ethyl acetate, washed once with a saturated sodium chloride solution, dried over magnesium sulphate, and evaporated under reduced pressure. The derivative obtained has been purified by silica gel chromatography using as the eluent: ethyl acetate/methanol/triethylamine in a gradient (98/0/2; 60/10/10; 70/20/10; 60/30/10).

The expected compound has been obtained in 53% yield as a yellow solid.

It has the following characteristics:

Melting point=86° C.

Molar mass=C₁₆H₃ON₄: 278.43 g/mol

¹H NMR (CD₃OD): 8.26 (d; J=4.8 Hz; 2H); 6.59 (t; J=4.9 Hz; 1H); 3.38-3.34 (m; 2H); 2.84-2.76 (m; 2H); 1.66-1.53 (m; 4H); 1.41-1.33 (m; 16H).

¹³C NMR (CD₃OD): 162.1; 157.9 (2C); 109.6; 40.8; 40.1; 29.3; 29.3; 29.3; 29.3; 29.2; 29.1; 29.1; 29.0; 26.6; 26.3.

### Second Step: Synthesis of the N′-(4,5-dihydro-1H-imidazol-2-yl)-N-pyrimidin-2-yl-dodecane-1,12-diamine derivative

To a solution of 100 mg (0.36 mmol) of N′-pyrimidin-2-yldodecane-1,12-diamine as prepared in the previous step in an ethanol/triethylamine mixture (12 mm/0.1 mm), 1.05 equivalent (0.38 mmol) of 1-(4,5-dihydro-1H-imidazol-2-yl)-3,5-dimethyl-1H-pyrazole hydrobromide has been added. The reaction medium has been heated under reflux of ethanol for 3 days. After cooling, the reaction mixture has been filtered and the filtrate concentrated in vacuo. The derivative obtained has been purified by silica gel chromatography using as the eluent: dichloromethane/methanol (92/8).

The expected compound has been obtained in 40% yield as a yellow solid.

It has the following characteristics:

Melting point=90° C.

Molar mass=C₁₉H₃₄N₆: 346.51 g/mol

¹H NMR (CD₃OD): 8.14 (d; J=4.9 Hz; 2H); 6.47 (t; J=4.8 Hz; 1H); 3.61 (s; 4H); 3.25-3.22 (m; 2H); 3.14-3.06 (m; 2H); 1.53-1.45 (m; 4H); 1.31-1.18 (m; 16H).

¹³C NMR (CD₃OD): 162.1; 159.9; 157.8 (2C); 109.6; 42.6; 42.5; 40.8; 40.5; 29.3; 29.2; 29.2; 29.2; 29.1; 29.1; 28.9; 28.8; 26.6; 26.3.

### Example 2: Synthesis of Compound Ib

Compound Ib has been prepared according to the steps illustrated in the following scheme:

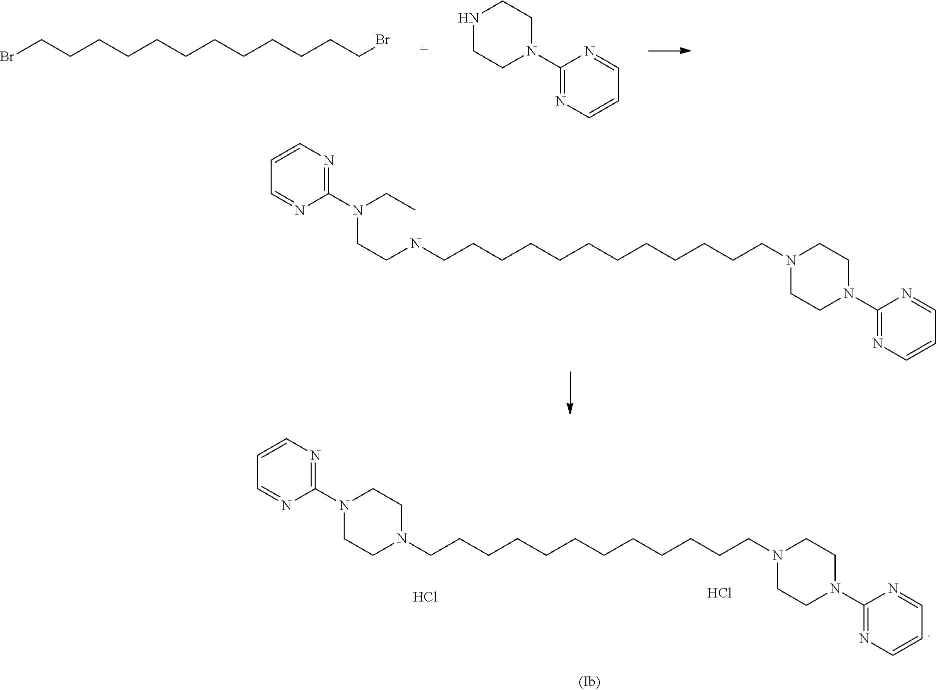 [SMILES: BrCCCCCCCCCCCCBr ; c1cnc(N2CCNCC2)nc1 ; CCN(CCNCCCCCCCCCCCCN1CCN(c2ncccn2)CC1)c1ncccn1 ; c1cnc(N2CCN(CCCCCCCCCCCCN3CCN(c4ncccn4)CC3)CC2)nc1 ; Cl ; Cl]

### First Step: Synthesis of the 2-[4-[12-(4-pyrimidin-2-ylpiperazin-1-yl)dodecyl]piperazin-1-yl]pyrimidine derivative

To a solution of 700 mg (2.13 mmole) of 1,12-dibromododecane in 30 mL of dioxane 2 equivalents (4.26 mmole; 700.5 mg) of 2-piperazin-1-ylpyrimidine and 7 equivalents (14.94 mmole; 2.1 g) of potassium carbonate have been added. The reaction mixture has been heated under reflux of dioxane for 20 hours. After cooling, the dioxane has been concentrated under reduced pressure. The residue obtained has been taken up in 50 mL of ethyl acetate, washed once with a saturated sodium chloride solution, dried over magnesium sulphate, and evaporated under reduced pressure. The derivative obtained has been purified by silica gel chromatography using as the eluent: ethyl acetate/methanol/triethylamine (99/0.5/0.5).

The expected compound has been obtained in 64% yield as a white solid.

It has the following characteristics:

Melting point=106° C.

Molar mass=C₂₈H₄₆N₈: 494.72 g/mol

¹H NMR (CD₃OD): 8.23 (d; J=4.7 Hz; 4H); 6.40 (t; J=4.7 Hz; 2H); 3.84-3.67 (m; 8H); 2.49-2.35 (m; 8H); 2.35-2.21 (m; 4H); 1.54-1.37 (m; 4H); 1.32-1.11 (m; 16H).

¹³C NMR (CD₃OD): 161.7 (2C); 157.7 (4C); 109.8 (2C); 59.0 (2C); 53.2 (4C); 43.7 (4C); 29.6 (δC); 27.6 (2C); 26.9 (2C).

### Second Step: Synthesis of the 2-[4-[12-(4-pyrimidin-2-ylpiperazin-1-yl)dodecyl]piperazin-1-yl]pyrimidine dihydrochloride derivative

150 mg (0.30 mmol) of 2-[4-[12-(4-pyrimidin-2-ylpiperazin-1-yl)clodecyl]piperazin-1-yl]pyrimidine have been dissolved in 5-10 mL of ethanol. Hydrochloric acid gas has been bubbled through the reaction medium for 5 minutes. After stirring, the precipitate has been collected by filtration, washed with diethyl ether and dried. The expected compound has been obtained in 87% yield as a white solid.

It has the following characteristics:

Melting point=252° C.

Molar mass=C₂₈H₄₆N₈·2HCl: 567.64 g/mol

¹H NMR (DMSO-d₆): 11.49 (s; 2H); 8.44 (d; J=4.8 Hz; 4H); 6.76 (t; J=4.8 Hz; 2H); 4.67 (d; J=14.1 Hz; 4H); 3.52 (d; J=12.4 Hz; 8H); 3.07-2.99 (m; 8H); 1.79-1.73 (m; 4H); 1.35-1.30 (m; 16H).

¹³C NMR (DMSO-d₆): 161.2 (2C); 158.5 (4C); 111.7 (2C); 56.2 (2C); 50.9 (4C); 40.8 (4C); 29.3 (2C); 29.1 (2C); 28.9 (2C); 26.6 (2C); 23.4 (2C).

### Example 3: Synthesis of Compound Ic

Compound Ic has been prepared according to the steps illustrated in the following scheme:

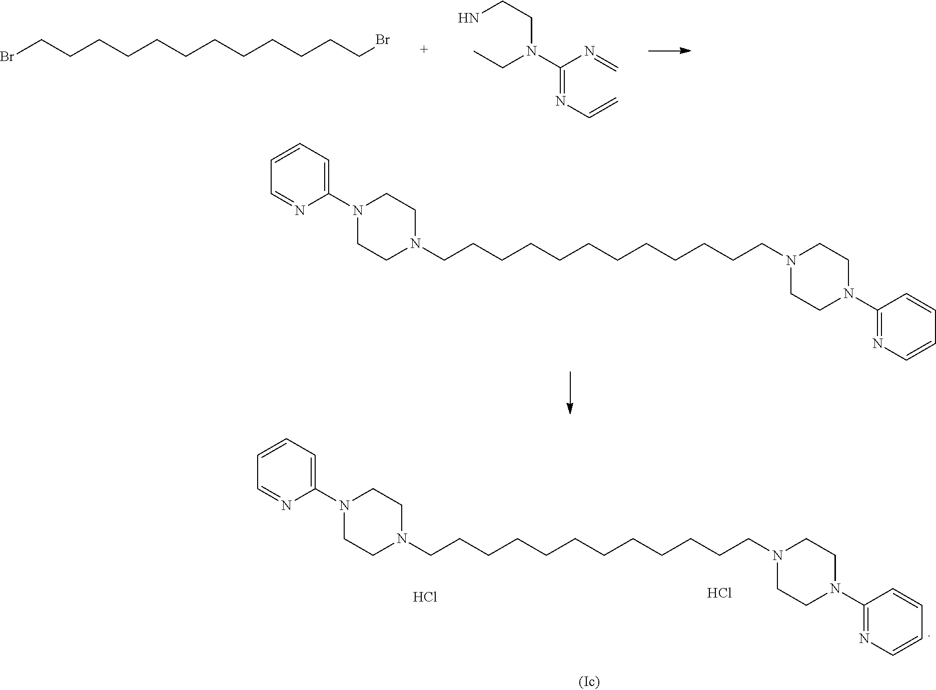 [SMILES: BrCCCCCCCCCCCCBr ; C=C/N=C(\N=C)N(CC)CCN ; c1ccc(N2CCN(CCCCCCCCCCCCN3CCN(c4ccccn4)CC3)CC2)nc1 ; c1ccc(N2CCN(CCCCCCCCCCCCN3CCN(c4ccccn4)CC3)CC2)nc1 ; Cl ; Cl]

### First Step: Synthesis of the 1-(2-pyridyl)-4-[12-[4-(2-pyridyl)piperazin-1-yl]dodecyl]piperazine derivative

To a solution of 700 mg (2.13 mmole) of 1,12-dibromododecane in 30 mL of dioxane, 2 equivalents (4.26 mmole; 696.3 mg) of 1-(2-pyridyl)piperazine and 7 equivalents (14.94 mmole; 2.1 g) of potassium carbonate have been added. The reaction mixture has been heated under reflux of dioxane for 20 hours. After cooling, the dioxane has been concentrated under reduced pressure. The residue obtained has been taken up in 50 mL of ethyl acetate, washed once with a saturated sodium chloride solution, dried over magnesium sulphate, and evaporated under reduced pressure. The derivative obtained has been purified by silica gel chromatography using as the eluent: ethyl acetate/methanol/triethylamine (99/0.5/0.5).

The expected compound has been obtained in 48% yield as a white solid.

It has the following characteristics:

Melting point=100° C.

Molar mass=C₃₀H₄₈N₆: 492.74 g/mol

¹H NMR (CD₃OD): 8.12 (ddd; J=4.9; 2.0; 0.9 Hz; 2H); 7.40 (ddd; J=8.9; 7.1; 2.0 Hz; 2H); 6.60-6.51 (m; 4H); 3.48 (dd; J=6.2; 4.1 Hz; 8H); 2.54-2.42 (m; 8H); 2.34-2.25 (m; 4H); 1.52-1.38 (m; 4H); 1.30-1.14 (m; 16H).

¹³C NMR (CD₃OD): 159.6 (2C); 148.0 (2C); 137.1 (2C); 113.2 (2C); 107.0 (2C); 59.0 (2C); 53.2 (4C); 45.2 (4C); 29.6 (δC); 27.6 (2C); 26.9 (2C).

### Second Step: Synthesis of the 1-(2-pyridyl)-4-[12-[4-(2-pyridyl)piperazin-1-yl]dodecyl]piperazine dihydrochloride derivative

150 mg (0.30 mmol) of 1-(2-pyridyl)-4-[12-[4-(2-pyridypiperazin-1-yl]dodecyl]piperazine have been dissolved in 5-10 mL of ethanol. Hydrochloric acid gas has been bubbled through the reaction medium for 5 minutes. After stirring, the precipitate has been collected by filtration, washed with diethyl ether and dried. The expected compound has been obtained in 91% yield as a white solid.

It has the following characteristics:

Melting point=200° C.

Molar mass=C₃₀H₄₈N₆·2HCl: 565.66 g/mol

¹H NMR (DMSO-d₆): 11.41 (s; 2H); 8.13 (dd; J=5.8; 1.8 Hz; 2H); 7.94 (t; 3=8.2 Hz; 2H); 7.30 (d; 3=9.0 Hz; 2H); 6.96 (t; 3=6.4 Hz; 2H); 4.48 (d; J=8.2 Hz; 4H); 3.63-3.57 (m; 8H); 3.05-3.17 (m; 8H); 1.82-1.68 (m; 4H); 1.31-1.28 (m; 16H). ¹³C NMR (DMSO-d₆) 155.3 (2C); 142.7 (2C); 141.7 (2C); 114.4 (2C); 110.9 (2C); 56.1 (2C); 50.5 (4C); 43.2 (4C); 29.3 (2C); 29.1 (2C); 28.9 (2C); 26.6 (2C); 23.4 (2C).

### Example 4 of the Use of Compounds (Ia), (Ib) and (Ic) as Cancer Agents

4.1 Seahorse Technology Analysis of Oxygen Consumption Rate after Treatment of Human B Cell Lymphoma Cells with Compounds (Ia), (Ib) and (Ic)

The analyser known as “Seahorse” allows evaluation mitochondrial respiration in real time by measuring Oxygen Consumption Rate (OCR). The experiments have been carried out according to the supplier's recommendations (“Agilent_Seahorse XFe96”). To achieve this, 150,000 cells per well of a human B cell lymphoma line (“RL line, CVCL_1660”) have been seeded in 180 μL of Agilent_Seahorse XF RPMI medium (supplemented with pyruvate and glucose) on a dedicated Agilent_Seahorse 96-well plate, pre-treated with a solution of CellTak according to the supplier's recommendations (Corning). After 30 min incubation in a CO₂-free oven, the plate has been analysed with the “Seahorse” using a protocol allowing injection (indicated by arrows in FIG. 1) of increasing amounts of compound to obtain a final cumulative concentration of 1.2 μM, 2.5 μM, 5 μM and 10 μM, followed each time by an OCR reading (in pmol/min) for 10 min. Data normalisation has been carried out by cell counting, using a device known as the “Cytation 1/5 Cell Imaging Multi-Mode reader” (BioTek Instruments, Inc) coupled to the “Seahorse”.

FIG. 1 shows the effect on mitochondrial respiration of several compounds: a reference inhibitor Rotenone (FIG. 1a, ROT, curve with inverted empty triangles), the inhibitor known as “IACS010759” currently being evaluated in clinical trials (FIG. 1a, IACS, curve with solid diamonds), compound (Ia) (FIG. 1b, curve with inverted solid triangles), compound (Ib) (FIG. 1b, curve with empty circles) and compound (Ic) (FIG. 1b, curve with solid circles). A control is used (DMSO, curve with solid squares) in each figure and corresponds to the medium studied without addition of compound (addition only of the DMSO diluent from the molecule stocks). In FIG. 1a, injection of a concentration of 1.2 μM induced instantaneous and almost complete inhibition of OCR, in a similar way for the two comparative compounds not in accordance with the invention: Rotenone and “IACS-010759”. In addition, injection of increasing concentrations of compounds in accordance with the invention induces progressive inhibition of OCR, in a similar manner and in stages for compounds (Ib) and (Ic), and progressively for compound (Ia). Virtually complete inhibition of OCR is obtained with a concentration of 10 μM.

In FIG. 1, the standard deviation (SD) is indicated.

In conclusion, compounds (Ia), (Ib) and (Ic) behave as mitochondrial respiration inhibitors (=also known as OXPHOS inhibitors), with a moderate and progressive effect compared to rotenone or “IACS-010759”. Equivalent results have been obtained for the various lines tested.

4.2 Analysis of the Effect on Cell Growth of Treatment of Human Tumour Cells from a B Cell Lymphoma Line with Compounds (Ia), (Ib) and (Ic)

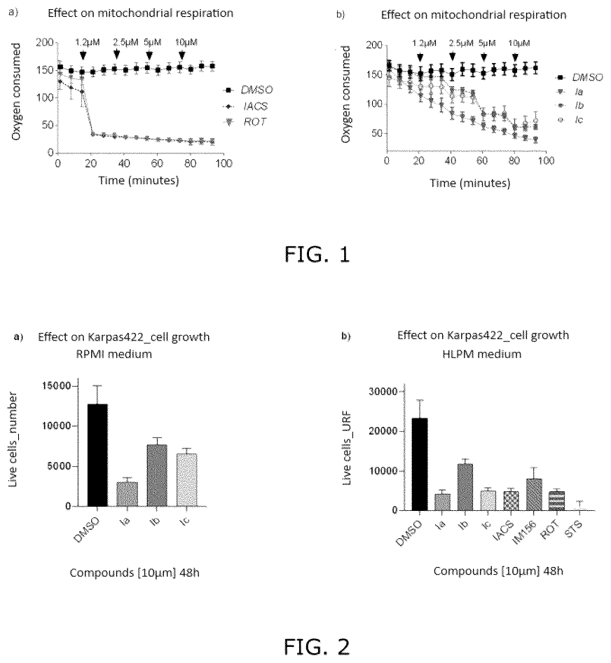

Cells from a human B cell lymphoma cell line (“Karpas422, CVCL_1325”) have been seeded at a density of 100,000 cells per well, in a 96-well plate in the presence of a 10 μM concentration of compound, in a volume of 100 μL of medium. After 48 h of treatment, analyses of the effect on cell growth have been conducted, either by counting live cells using flow cytometry (FIG. 2a), or using a biochemical fluorescence detection kit for live cells (FIG. 2b).

FIG. 2 shows the effect on cell growth of treating cells in a conventional “Gibco RPMI1640/10% SVF” medium (denoted RPMI), with compounds (Ia), (Ib), and (Ic) in accordance with the invention (FIG. 2a); and in a “Gibco Human Like Plasma Medium” (Gibco HPLM_Thermo Fisher Scientific) (noted HLPM), mimicking the metabolic conditions of human plasma, with compounds (Ia), (Ib), and (Ic) in accordance with the invention and with four comparative compounds not in accordance with the invention: “IACS-010759 (IACS), an inhibitor known as “IM156” currently in clinical trials, Rotenone (ROT), and an apoptosis inducer, Staurosporine (STS) (FIG. 2b). In conventional “Gibco RPMI1640/10°/0 SVF” medium, the number of living cells has been determined after double AnnexinV/PI labelling, and analysis using the flow cytometer known as “ATTUNE” (Thermo Fisher Scientific). In “Gibco Human Like Plasma Medium”, the number of live cells has been evaluated using the “CellTiterFluor” kit and after measurement of relative fluorescence units (RFU) according to the supplier's recommendations (Promega). The term DMSO in FIG. 2 corresponds to the control, i.e. identical medium with no added compound.

In FIG. 2, the standard deviation (SD) is indicated.

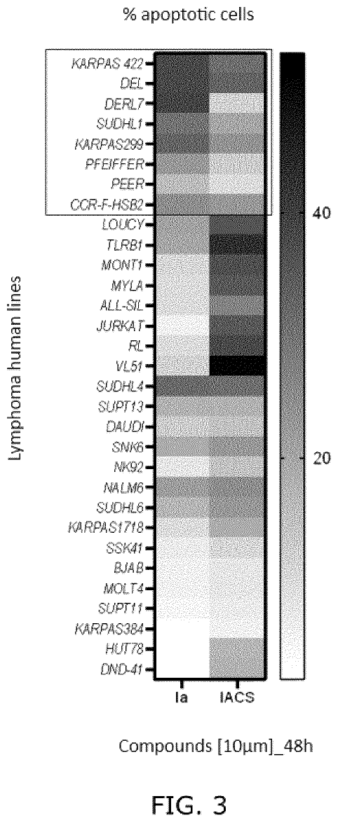

Based on FIG. 2, compounds (Ia), (Ib) and (Ic) inhibit cell growth by reducing the number of live tumour cells after 48 hours of treatment, even in culture medium conditions mimicking human plasma. The efficacy is similar to that obtained with the inhibitor “IACS010759” or rotenone. This effect is line-dependent (see FIG. 3).

4.3 Analysis of the Effect on Cell Growth of Treating Human Tumour Cells from a Wide Series of B and T Cell Lymphoma Cell Lines with Compound (Ia)

Analyses of the effect on cell growth of compound (Ia) in accordance with the invention in comparison with compound “IACS-010769” (denoted IACS) not in accordance with the invention have been conducted on a wide series of 31 human B and T cell lymphoma lines, after AnnexinV/PI labelling and analysis with the “ATTUNE” cytometer (Thermo Fisher Scientific). In addition to the number of living cells represented in FIG. 2a, this technology enables the % of cells undergoing apoptosis to be evaluated. The results have been represented in FIG. 3 in the form of a “HeatMap” with a black and white gradient, black representing the highest % of apoptotic cells.

Based on FIG. 3, the toxicity of compounds (Ia) and “IACS-010759” varies according to the lymphoma lines analysed. In particular, the toxicity of compound (Ia) is increased with respect to that of “IACS-010759” for a sub-group of 8 lines framed in the diagram. These data confirm that these 2 compounds have a different metabolic targeting and mode of action.

### 4.4 Analysis of the Antitumour Efficacy of Compound (Ia) in an in Ovo Model

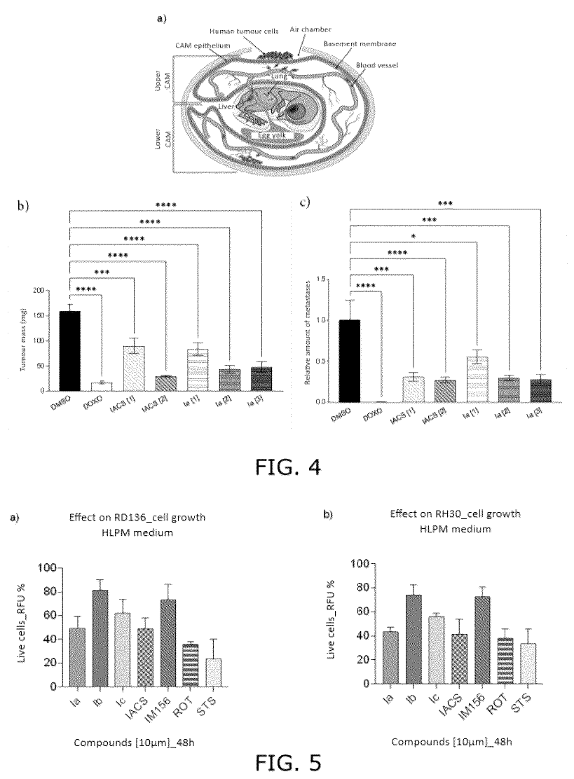

Preclinical evaluation analyses of the anti-tumour efficacy of compound (Ia) have been carried out using the xenograft model on the chorioallantoic membrane (CAM) of the chicken embryo. FIG. 4 shows the implantation of human tumour cells of a B cell lymphoma line (“SUDHL-4, CVCL_0539”) on the upper CAM of an embryo at day E9 (FIG. 4a), as well as the indication of presence or absence of a tumour mass (FIG. 4b), and the relative amount of metastasis (FIG. 4c). Treatments have been performed on days E11, E13, E15 and E17. At day E18, the upper CAM portions containing tumours have been removed and weighed, and the lower CAM fragments have been used to evaluate the presence of human cells indicative of metastases by RT-gPCR (polymerase chain reaction from an RNA sample). 0.0097 mg/kg Doxorubicin (DOXO) has been used as a positive control for treatment efficacy, compound (Ia) has been used at 3 different doses [1]: 0.05 mg/kg, [2]: 0.15 mg/kg, and [3]: 0.45 mg/kg, and “IACS-010759” has been used at 2 different doses [1]: 0.05 mg/kg, and [2]: 0.45 mg/kg. The negative control (denoted DMSO) represents treatment with the DMSO molecule diluent.

In FIG. 4b, the effect of treatment on tumour volume is directly observed. Treatment with doxorubicin induced a 90% reduction in primary tumour volume. In comparison, treatment with “IACS010759” (IACS) induced a 44% [1] and 82% [2] reduction, and treatment with compound (Ia) induced a 48% [1], 73% [2] and 70% [3] reduction.

In FIG. 4c, the effect of treatment on the relative number of metastases is directly observed. Treatment with doxorubicin induced a 99% regression in the number of metastases. In comparison, treatment with “IACS 010759” (IACS) induced a 69% [1] and 73% [2] reduction, and treatment with compound (Ia) induced a 45% [1], 70% [2] and 72% [3] reduction.

In FIG. 4, the standard deviation (SD) is indicated.

In conclusion, compound (Ia), which is chemically different and acts differently on the OCR, has antitumour efficacy comparable to that of the “IACS-010759” molecule, in the preclinical model used in this example. Compound (Ia) also inhibits tumour dissemination.

4.5 Analysis of the Effect on Cell Growth of Treating Human Cells from Two Sarcoma Lines with Compounds (Ia), (Ib) and (Ic)

Human tumour cells from two paediatric rhabdomyosarcoma-like sarcoma lines (“RD136, CVCL_1649”, FIG. 5a and “RH3O, CVCL_0041”, FIG. 5b) have been seeded in a 96-well plate in the presence of a concentration of 10 μM of compound, in a volume of 100 μL of “Gibco Human Like Plasma Medium” (Gibco HPLM_Thermo Fisher Scientific). The effect of compounds (Ia), (Ib) and (Ic) has been compared with that of the inhibitors IACS-010759 (IACS) and IM156 (metformin analogue) currently in clinical trials, Rotenone (ROT) and the apoptosis inducer staurosporine (STS). After 48 hours of treatment, the effect on the number of living cells has been evaluated using the “CellTiterFluor” kit from Promega, identically to FIG. 2b.

FIG. 5a shows the effect of treatment by the compounds on the number of live cells in RD136 line (in HLPM medium as defined above) and FIG. 5b the effect on the number of live cells in RH30 line (in HLPM medium as defined above), represented as % relative fluorescence units (RFU) compared to the effect of compound diluent (DMSO).

In FIG. 5, the standard deviation (SD) is indicated.

In conclusion, compounds (Id), (Ib) and (Ic) inhibits cell growth in the RD136 and RH30 lines by reducing the number of living cells after 48 h of treatment. Their effect is similar to that obtained with the inhibitor “IACS-010759” or rotenone. The field of application for these compounds can therefore be extended beyond haematological cancers such as lymphomas, for example to solid tumours such as the paediatric human sarcomas exemplified here.

4.6 Analysis of the IC₅₀ of a Compound (Ia) in Accordance with the Invention Compared with that of a Compound of Prior Art

The IC₅₀ of compound (Ia) has been compared with that of a compound as described in WO2020/109506 having the following formula:

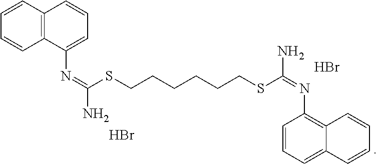 [SMILES: N/C(=N/c1cccc2ccccc12)SCCCCCCS/C(N)=N\c1cccc2ccccc12 ; Br ; Br]

The results have shown an IC₅₀ of 1.5 μM for compound (Ia) and 15.1 μM for the compound as described in WO2020/109506. This shows the improved performance of compound (Ia) compared to the compound of prior art. These experiments have been performed using cells from a human B cell lymphoma cell line (“Karpas422, CVCL_1325”) which have been seeded in “RPMI 1640” medium (11 mM glucose) supplemented with 10% serum, in the presence of increasing concentrations of compounds.

After 48 h of treatment, analyses of the effect on cell growth have been conducted, using the “CellTiter-Fluor Cell Viability Assay” (Promega) live cell detection kit, as in FIG. 2b, and following the supplier's instructions.

### Example 5: Synthesis of Compound Id

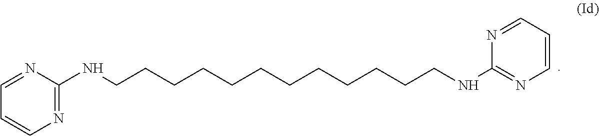 [SMILES: c1cnc(NCCCCCCCCCCCCNc2ncccn2)nc1]

To a solution of 700 mg (3.49 mmoles) of 1,12-dodecanediamine in 30 mL of dioxane, 2.2 equivalents (7.69 mmoles; 880 mg) of 2-chloropyrimidine and 5 equivalents (17.47 mmoles; 2.4 g) of potassium carbonate have been added. The reaction mixture has been heated under reflux of dioxane for 20 h. After cooling, the dioxane has been concentrated under reduced pressure. The residue obtained has been taken up in 50 mL of ethyl acetate, washed once with a saturated sodium chloride solution, dried over magnesium sulphate, and evaporated under reduced pressure. The derivative obtained has been purified by silica gel chromatography using as the eluent: ethyl acetate/cyclohexane (90/10).

The expected compound has been obtained in 26% yield as a white solid.

It has the following characteristics: melting point Mp=105° C., C₂₀H₃₂N₆=356.50 g/mol.

¹H NMR (CDCl₃): 8.20 (d; J=4.8 Hz; 4H); 6.43 (t; J=4.8 Hz; 2H); 5.11 (bs; 2H); 3.32 (td; J=7.1; 5.7 Hz; 4H); 1.57-1.50 (m, 4H); 1.35-1.18 (m; 16H).

¹³C NMR (CDCl₃): 162.4 (2C); 158.0 (4C); 110.3 (2C); 41.5 (2C); 29.6 (2C); 29.5 (2C); 29.4 (2C); 27.0 (2C)

### Example 6: Synthesis of the Compound Ie

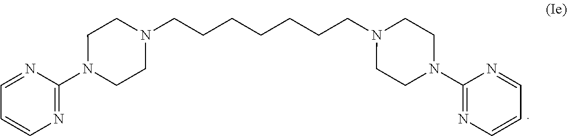 [SMILES: c1cnc(N2CCN(CCCCCCCN3CCN(c4ncccn4)CC3)CC2)nc1]

To a solution of 800 mg (3.10 mmoles) of 1,7-dibromoheptane in 30 mL of dioxane 2 equivalents (6.20 mmoles; 1.0 g) of 2-piperazin-1-ylpyrimidine and 7 equivalents (21.70 mmoles; 3.0 g) of potassium carbonate have been added. The reaction mixture has been heated under reflux of dioxane for 20 hours. After cooling, the dioxane has been concentrated under reduced pressure. The residue obtained has been taken up in 50 mL of dichloromethane, washed once with a saturated sodium chloride solution, dried over magnesium sulphate, and evaporated under reduced pressure. The derivative obtained has been purified by silica gel chromatography using as the eluent: dichloromethane/methanol/triethylamine (98/1/1).

The expected compound has been obtained in 30% yield as a white solid.

It has the following characteristics: melting point Mp=86° C., C₂₃H₃₆N₈=424.58 g/mol.

¹H NMR (CDCl₃): 8.23 (d; J=4.7 Hz; 4H); 6.40 (t; J=4.7 Hz; 2H), 3.78-3.75 (m; 8H); 2.44-2.41 (m; 8H); 2.31-2.27 (m; 4H); 1.50-1.43 (m; 4H); 1.28-1.24 (m; δH).

¹³C NMR (CDCl₃): 161.7 (2C); 157.7 (4C); 109.8 (2C); 58.9 (2C); 53.2 (4C); 46.2 (2C); 43.7 (2C); 29.5; 27.6 (2C); 26.9 (2C).

### Example 7: Synthesis of the Compound If

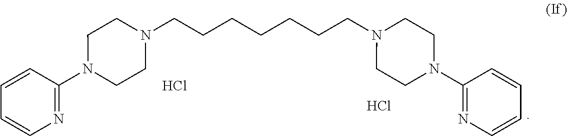 [SMILES: c1ccc(N2CCN(CCCCCCCN3CCN(c4ccccn4)CC3)CC2)nc1 ; Cl ; Cl]

To a solution of 800 mg (3.10 mmoles) of 1,7-dibromoheptane in 30 mL of dioxane, 2 equivalents (6.20 mmoles; 1.0 g) of 1-(2-pyridyl)piperazine and 7 equivalents (21.70 mmoles; 3.0 g) of potassium carbonate have been added. The reaction mixture has been heated under reflux of dioxane for 20 hours. After cooling, the dioxane has been concentrated under reduced pressure. The residue obtained has been taken up in 50 mL of dichloromethane, washed once with a saturated sodium chloride solution, dried over magnesium sulphate, and evaporated under reduced pressure. The derivative obtained has been purified by silica gel chromatography using as the eluent: dichloromethane/methanol/triethylamine (98/1/1).

150 mg (0.354 mmole) of 1-(2-pyridyl)-4-[7-[4-(2-pyridypiperazin-1-yl]heptyl]piperazine have been dissolved in 5-10 mL of ethanol. Hydrochloric acid gas has been bubbled through the reaction medium for 2 minutes. After stirring, the precipitate has been collected by filtration, washed with diethyl ether, and dried.

The expected compound has been obtained in 82% yield as a white solid.

It has the following characteristics: melting point Mp=210° C., C₂₅H₃₈N₆·2HCl=495.53 g/mol.

¹H NMR (D₂O): 8.06 (m; 2H); 7.93 (d; J=6.3 Hz; 2H); 7.30 (d; J=9.2 Hz; 2H); 7.06 (t; J=6.7 Hz; 2H); 4.28 (d; J=14.5 Hz; 4H); 3.73 (d; J=12.8 Hz; 4H); 3.59 (t; J=13.5 Hz; 4H); 3.30-3.13 (m; 8H); 1.71 (m; 4H); 1.34 (m; 6H).

¹³C NMR (D₂O): 152.1 (2C); 145.6 (2C); 136.6 (2C); 115.0 (2C); 113.3 (2C); 57.1 (2C); 50.5 (4C); 43.2 (4C); 27.7; 25.5 (2C); 23.3 (2C).

### Example 8 of Use of Compounds (Id), (Ie) and (If) as Cancer Agents

Cells from the human B cell lymphoma cell line (“RL, CVCL_1660”), have been seeded at a density of 100,000 cells per well, in a 96-well plate in the presence of a concentration of 5 μM or 10 μM of compound, in a volume of 100 μL of RPMI medium. After 48 h of treatment, analyses of the effect on cell growth have been conducted by counting live cells using flow cytometry, as in FIG. 2a.

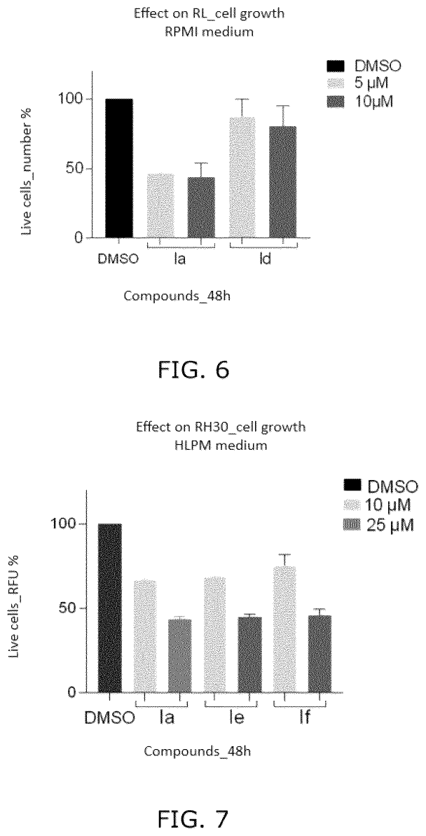

FIG. 6 shows the inhibitory effect on cell growth of treating cells in a conventional “Gibco RPMI1640/10% SVF” medium (denoted RPMI), with compounds (Ia) and (Id) in accordance with the invention.

In FIG. 6, the standard deviation (SD) is indicated.

Human tumour cells from the paediatric rhabdomyosarcoma sarcoma line (“RH30, CVCL_0041”) have been seeded in a 96-well plate in the presence of a 10 μM or 25 μM concentration of compound, in a 100 μL volume of “Gibco Human Like Plasma Medium” (Gibco HPLM_Thermo Fisher Scientific). After 48 h of treatment, the effect of compounds (Ia), (Ie) and (If) on cell growth has been evaluated using the Promega “CellTiterFluor” kit as in FIG. 2b. FIG. 7 shows the effect of treatment by the compounds on RH30 line growth (in HLPM medium as defined above), represented as % relative fluorescence units (RFU) compared to the effect of the compound diluent (DMSO).

In FIG. 7, the standard deviation (SD) is indicated.

In conclusion, compounds (Ia), (Ie) and (If) inhibit cell growth of the RH30 line by reducing the number of living cells after 48 h of treatment. The field of application for these compounds can therefore be extended beyond haematological cancers such as lymphomas, for example to solid tumours such as the paediatric human sarcomas exemplified here.

1-15. (canceled)

16. A compound selected from compounds having the formula (I), pharmaceutically acceptable salts thereof and pharmaceutically acceptable solvates thereof, said formula (I) having the following structure: 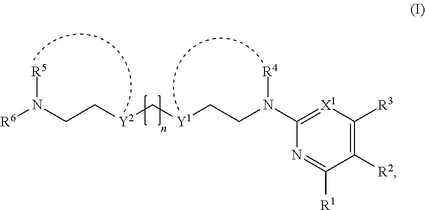
wherein:
R¹, R², and R³ represent, independently of each other, a hydrogen atom, a halogen atom, an alkyl radical, a cycloalkyl radical, an aryl radical, or a group selected from —OH, NH₂, —SH, —CN, —CF₃, —CHO, —NH—NH₂, —CO₂H, —CH₂OH, —CH₂NH₂, an alkoxy group —OR⁷, a —NR⁸R⁹ group, a —SR¹⁰ group, a —C(O)R¹¹ group, a —CH₂OR¹² group, and a —CH₂NR¹³R¹⁴ group, with R⁷, R⁸, R¹⁰, R¹¹, R¹² and R¹³ representing, independently of each other, an alkyl or cycloalkyl radical, and R⁹ and R¹⁴ representing, independently of each other, a hydrogen atom or an alkyl or cycloalkyl radical,
X¹ represents a nitrogen atom or a CR¹⁵ group, with R¹⁵ being a hydrogen atom, a halogen atom, an alkyl radical, a cycloalkyl radical, an aryl radical, or a group selected from —OH, NH₂, —SH, —CN, —CF₃, —CHO, —NH—NH₂, —CO₂H, —CH₂OH, —CH₂NH₂, an alkoxy group —OR⁷, a —NR⁸R⁹ group, a —SR¹⁰ group, a —C(O)R¹¹ group, a —CH₂OR¹² group, and a —CH₂NR¹³R¹⁴ group,
n is an integer ranging from 1 to 20,
R⁴ represents a hydrogen atom, an alkyl radical, or a divalent alkylene group forming a ring with Y¹ and the nitrogen atom to which R⁴ is attached,
R⁵ represents a hydrogen atom, an alkyl radical, or a divalent alkylene group forming a ring with Y² and the nitrogen atom to which R⁵ is attached,
Y¹ represents —CH₂—, —NH—, or —O— when the R⁴ group represents a hydrogen atom or an alkyl radical; and represents —CH— or —N— when the R⁴ group represents a divalent alkylene group forming a ring with Y¹ and the nitrogen atom to which R⁴ is attached,
Y² represents —CH₂—, —NH— or —O— when the R⁵ group represents a hydrogen atom or an alkyl radical; and represents —CH— or —N— when the R⁵ group represents a divalent alkylene group forming a ring with Y² and the nitrogen atom to which R⁴ is attached, and
R⁶ represents one of the following two groups (IIa) and (IIb): 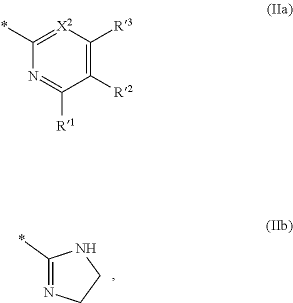
wherein R′¹, R′², and R′³ represent, independently of each other, a hydrogen atom, a halogen atom, an alkyl radical, a cycloalkyl radical, an aryl radical, or a group selected from —OH, —NH₂, —SH, —CN, —CF₃, —CHO, —NH—NH₂, —CO₂H, —CH₂OH, —CH₂NH₂, an alkoxy group —OR′⁷, a —NR′⁸R′⁹ group, a —SR′¹⁰ group, a —C(O)R′¹¹ group, a —CH₂OR′¹² group, and a —CH₂NR′¹³R′¹⁴ group, with R′⁷, R′⁸, R′¹⁰, R′¹¹, R′¹², and R′¹³ representing, independently of each other, an alkyl or cycloalkyl radical, and R′⁹ and R′¹⁴ representing, independently of each other, a hydrogen atom or an alkyl or cycloalkyl radical, and
X² represents a nitrogen atom or a CR′¹⁵ group, with R′¹⁵ being a hydrogen atom, a halogen atom, an alkyl radical, a cycloalkyl radical, an aryl radical, or a group selected from —OH, —NH₂, —SH, —CN, —CF₃, —CHO, —NH—NH₂, —CO₂H, —CH₂OH, —CH₂NH₂, an alkoxy group —OR′⁷, a —NR′⁸R′⁹ group, a —SR′¹⁰ group, a —C(O)R′¹¹ group, a —CH₂OR′¹² group, and a —CH₂NR′¹³R′¹⁴ group,
for use in cancer treatment.

17. The compound as defined in claim 16, for use in the treatment of cancers with altered metabolism, in particular in the treatment of cancers with OXPHOS metabolism.

18. The compound as defined in claim 16, for use in the treatment of lymphomas; solid tumours; and some recurrent tumours following chemotherapy treatments.

19. The compound as defined in claim 16, wherein X¹ represents a nitrogen atom or a CH group.

20. The compound as defined in claim 16, wherein R⁴ represents a hydrogen atom or a divalent alkylene group forming a ring with Y¹ and the nitrogen atom to which R⁴ is attached, and R⁵ represents a hydrogen atom or a divalent alkylene group forming a ring with Y² and the nitrogen atom to which R⁵ is attached.

21. The compound as defined in claim 16, wherein Y¹ represents —CH₂— when the R⁴ group represents a hydrogen atom, or an alkyl radical; and represents —N— when the R⁴ group represents a divalent alkylene group forming a ring with Y¹ and the nitrogen atom to which R⁴ is attached; and Y² represents —CH₂— when the R⁵ group represents a hydrogen atom or an alkyl radical; and represents —N— when the R⁵ group represents a divalent alkylene group forming a ring with Y² and the nitrogen atom to which R⁵ is attached.

22. The compound as defined in claim 16, wherein Y¹ and Y² are identical.

23. The compound as defined in claim 16, wherein:
when Y¹ represents —CH₂—, n ranges from 2 to 10,
when Y² represents —CH₂—, n ranges from 2 to 10,
when Y¹ represents —N—, —NH—, —CH— or —O—, n ranges from 6 to 18, and
when Y² represents —N—, —NH—, —CH— or —O—, n ranges from 6 to 18.

24. The compound as defined in claim 16, wherein when Y¹ and Y² represent —CH—, and R⁶ is a group of formula (IIa), then n is such as n≥5 and/or R¹, R′¹, R², R′², R³, and R′³ are different from —NH₂; and when Y¹ and Y² represent —N—, and R⁶ is a group of formula (IIa), then n is such as n≥7.

25. The compound as defined in claim 16, wherein R⁶ is a group of formula (IIb).

26. The compound as defined in claim 16, wherein the compound of formula (I) is selected from the following compounds:

| Name | Structural formula |
|---|---|
| Compound Ia | 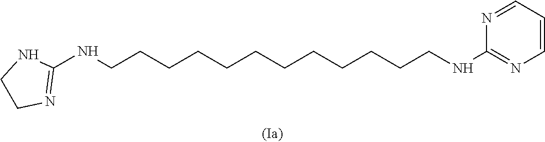 [SMILES: c1cnc(NCCCCCCCCCCCCNC2=NCCN2)nc1] |
| Compound Ib | 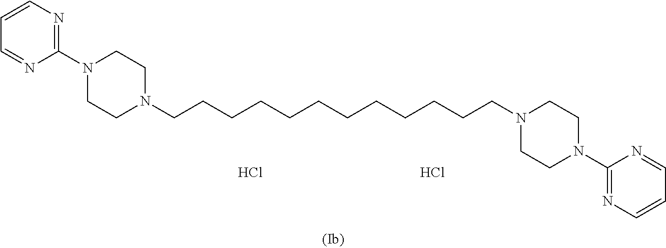 [SMILES: c1cnc(N2CCN(CCCCCCCCCCCCN3CCN(c4ncccn4)CC3)CC2)nc1 ; Cl ; Cl] |
| Compound Ic | 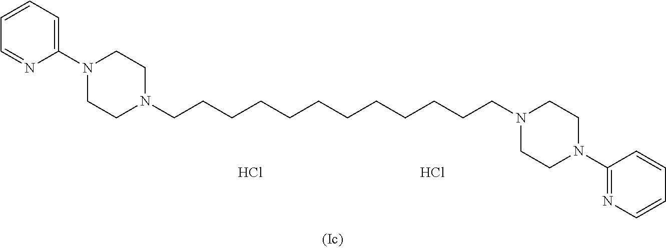 [SMILES: c1ccc(N2CCN(CCCCCCCCCCCCN3CCN(c4ccccn4)CC3)CC2)nc1 ; Cl ; Cl] |
| Compound Id | 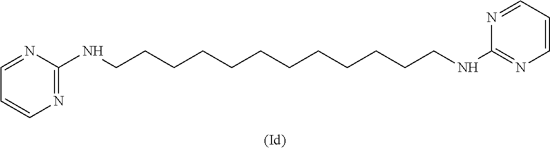 [SMILES: c1cnc(NCCCCCCCCCCCCNc2ncccn2)nc1] |
| Compound Ie | 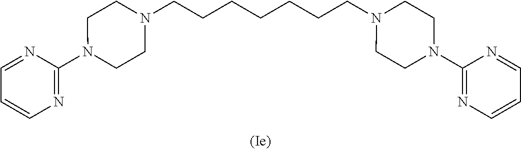 [SMILES: c1cnc(N2CCN(CCCCCCCN3CCN(c4ncccn4)CC3)CC2)nc1] |
| Compound If | 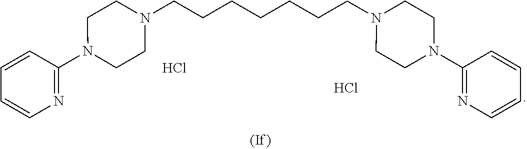 [SMILES: c1ccc(N2CCN(CCCCCCCN3CCN(c4ccccn4)CC3)CC2)nc1 ; Cl ; Cl ; "."] |

27. A pharmaceutical composition comprising the compound as defined in claim 16, and at least one suitable pharmaceutical carrier.

28. A product comprising the compound as defined in claim 16, and another active agent.

29. A compound selected from compounds having the formula (I′), pharmaceutically acceptable salts thereof, and pharmaceutically acceptable solvates thereof,
said formula (I′) having the following structure: 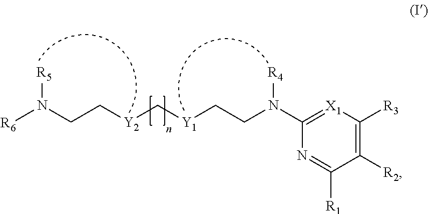
wherein:
R¹, R², R³, R⁴, R⁵, R⁶, X¹, Y¹, Y², and n are as defined in claim 16, wherein:
when Y¹ and Y² represent —CH—, and R⁶ is a radical of formula (IIa), then n≥5 and/or R¹, R′¹, R², R′², R³, and R′³ are different from —NH₂, and
when Y¹ and Y² represent —N—, and R⁶ is a radical of formula (IIa), then n≥7.

30. The compound according to claim 29, wherein R⁶ is a group of formula (IIb).

31. A method of treating a cancer in an individual, comprising administering to said individual a therapeutically effective amount of the compound according to claim 16.

32. The method as defined in claim 31, wherein the cancer has altered metabolism.

33. The method as defined in claim 32, wherein the altered metabolism is OXPHOS metabolism.

34. The compound as defined in claim 31, wherein the cancer is selected from the group consisting of a lymphoma; solid tumour; and recurrent tumours following chemotherapy treatments.
